(19) United States
(12) Patent Application Publication
(10) Pub. No.: US 2024/0270666 A1
(43) Pub. Date: Aug. 15, 2024

(54) SYMMETRIC TETRAALKYNYLATED ANTHRACENES AND THE PROCESS FOR PREPARING THE SAME FOR SENSING AND OPTOELECTRONIC APPLICATIONS

(71) Applicant: INDIAN INSTITUTE OF TECHNOLOGY, GUWAHATI, Guwahati (IN)

(72) Inventors: Akshai KUMAR, Guwahati (IN); Roy Paily PALATHINKAL, Guwahati (IN); Khadimul ISLAM, Guwahati (IN); Thomas T DANIEL, Guwahati (IN)

(21) Appl. No.: 18/681,039

(22) PCT Filed: Aug. 3, 2022
(86) PCT No.: PCT/IB2022/057185

§ 371 Date: Feb. 3, 2024

(30) Foreign Application Priority Data
Aug. 3, 2021 (IN) 202131035020

(51) Int. Cl. C07C 15/28; C07C 7/12; C07C 49/217; C07C 211/50; C07C 211/54; C07D 409/14; C07F 7/08; H10K 85/60

(52) U.S. Cl. CPC C07C 15/28; C07C 7/12; C07C 49/217; C07C 211/50; C07C 211/54; C07D 409/14; C07F 7/0805; H10K 85/615; C07C 2603/24

(57) ABSTRACT

The present invention relates to symmetric tetraalkynylated anthracene and more particularly tetraethynylated compounds with Formula III. The invention also provides a tetrafold sonogashira route towards these of symmetric tetraethynylated anthracene compounds with Formula III. The compounds of the present invention show good to excellent synthetic yield and find application in sensors and optoelectronic devices and show positive solvatochrism and halochromism. The sensor uses symmetric tetraalkynylated anthracene as channel material and the sensor has high sensitivity of 19.95 percent to 900 ppm and 0.86 percent to 50 ppm.

### FIELD OF THE INVENTION

The present invention relates to symmetric tetraalkynylated anthracenes which exhibit photophysical properties. More particularly, the present invention relates to symmetric tetraethynylated anthracene compounds of Formula III and a novel process for synthesizing symmetric tetraethynylated anthracene compounds of Formula III.

### BACKGROUND OF THE INVENTION

The immense development in the field of organic fluorescence especially organic light-emitting diodes (OLEDs), organic field-effect transistors (OFETs), solar cells, and sensors, and have gained tremendous attention owing to their outstanding performance efficiency and an economic solution to other light displays.

Polyalkylynation of polyaromatic hydrocarbons (PAHs) having aromatic nucleus like benzene, naphthalene, anthracene, pyrene, perylenes and chrysene have found application in the field organic fluorescence as they offer dynamic scope in tuning the photo physical properties of these compounds. Polyalkylynation of the PAHs not only improves the π-conjugation but also significantly improves their thermal stability paving ways for fabrication in optoelectronic devices with tunable properties.

Anthracenes have been known for longer time as blue time emitting compounds and possess high fluorescence and quantum yield because of its highly conjugated and rigid structure.

Steady state and time resolved fluorescence emission properties of symmetrical dialkoxyanthracenes (especially substituted on the side rings) have been reported by T. brotin et al (Photochemistry and Photobiology Vol. 55, No. 3, pp. 349-358. 1992).

The US20050233165 disclosed an anthracene derivative represented by the following general Formula I which enables an organic electroluminescence device to exhibit a great efficiency of light emission and uniform light emission even at high temperatures.

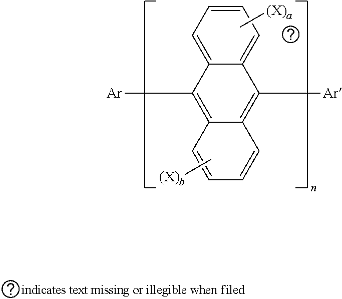

Ar represents a group represented by the following general Formula II:

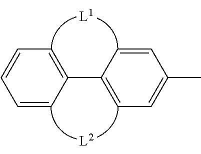

Another US2008475940 disclosed an anthracene derivative with Formula I which are used in Light emitting device.

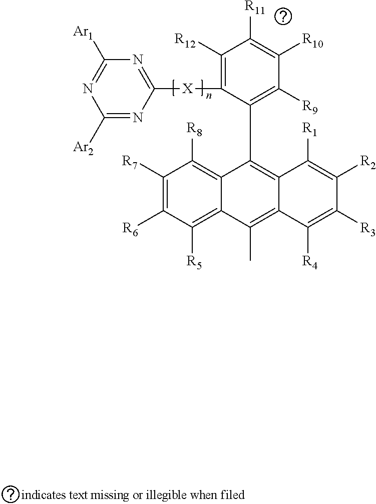

Park et al; (J.-H. Park et al./Organic Electronics 11 (2010) 820-830); discloses preparation and characterization of single crystals and thin films composed of three new small molecules based on soluble triisopropylsilylethynyl (TIPS)-substituted anthracene (TIPSAnt) for use in organic thin-film transistors (TFTs). The disclosed molecules are TIPSAnt derivatives containing thiophene (TIPSAntT), benzothiophene (TIPSAntBeT), or phenyl thiophene (TIPSAntPT) end cappers. The synthesis of the above compounds was carried out using tetrakis(triphenyl phosphine)palladium(0), sodium carbonate and phase transfer catalyst Aliquat-336 in THF as solvent and the reaction was carried out for 3 days.

Choi et al discloses synthesis unsymmetrical 2, 6, 9, 10-tetraalkynylatedanthracene molecules by two-step. A) synthesis of the symmetrical dial-kynylarylanthracene-9, 10-dione starting from 2, 6-dibromoanthracene-9, 10-dione via a Sonogashira coupling. B) Introduction of second pair of alkynyl groups to 2, 6-dialkynylaryl-9, 10-dione by the treatment of appropriate alkynes in the presence of SnCl2 and HCl leading to 2, 6, 9, 10-tetraalkynylatedanthracene.

PAHs with anthracene core that has three rigid fused aromatic ring containing 14π-electron that typically has a bandgap of 3.9 eV and gives high fluorescence quantum yield owing to optimal vibronic energy level. PAHs with anthracene core offer wider scope for optimization and derivatization which would be helpful in tuning Photophysical Properties of these Anthracenes. The design and synthesis of tetraalkynylated anthracene derivate with desired properties and convenient and robust Synthetic approach still remains unexplored. The current state of art focus mainly on non-symmetric anthracene derivate, and further the process involved in synthesis of tetraalkynalated anthracenes are tedious and require longer duration for completion.

None of the prior arts either disclose symmetric tetraalkynalated anthracenes nor any synthetic route for development of such compounds. In light of the above, there exists a need to explore new anthracene derivatives and substantially the new approaches for synthesizing these derivatives. The present invention is an endeavor in this direction.

### Object of the Invention

The main object of the present invention is to provide a novel process for synthesis of symmetric tetraalkynylated anthracenes with Formula III by tetrafold sonogashira coupling.

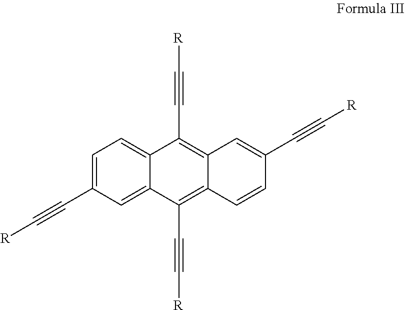

Another object of the present invention is to provide a single step one pot route for synthesis of the symmetric tetraalkynylated anthracenes.

Yet another object of the present invention is to provide new symmetric tetraethynylated anthracenes which show positive solvatochrism and halochromism.

Yet another object of the present invention is to explore the photophysical properties of these symmetric tetraethynylated anthracenes.

Yet another object of the present invention is to provide crystallographically characterized tetraalkynylated anthracenes.

Still another object of the present invention is to provide anthracene derivatives which find applications in sensors and optoelectronic devices.

### SUMMARY OF THE INVENTION

This summary is only intended to provide an introduction of the invention and does not determine the scope of the invention. This summary only introduces the aspects of the invention in a simpler form.

The present invention relates to symmetric tetraalkylynated anthracenes and more particularly to tetraethynylated anthracenes and a novel process for synthesis of symmetric tetraethynylated anthracenes of Formula III;

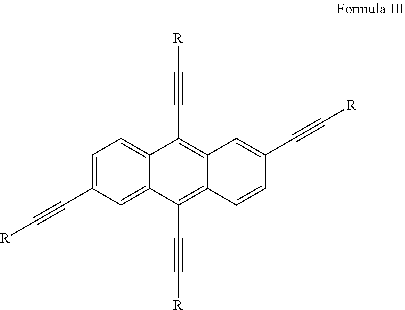

Where R is hydrogen, alkyl, halo, aryl, substituted aryl, hetroaryl;
alkyl is selected from methyl, ethyl, isopropyl, cyclopentyl, cyclohexyl, amino, N—N dimethyl, trimethyl silyl, trifluoromethyl;
Aryl is selected from phenyl, Chloro phenyl, bromo phenyl, methyl benzene, ethyl benzene, methoxy benzene, N—N dimethyl aniline, toluly, 1-Napthyl, anthracenyl, biphenyl, benzophenone, triphenyl amine.
Heteroaryl group is selected from thiophene, furan, pyrole, pyridine, imidiazole, benzimidazole, quinolone, isoquinoline.
Halo is selected from chloro, Bromo, fluoro.

In an embodiment, the present invention provides a direct single-step one-pot route to synthesis of symmetric tetraethynylated anthracene compounds with Formula III.

In another embodiment, the present invention provides a general scheme for synthesis of compounds with Formula III;

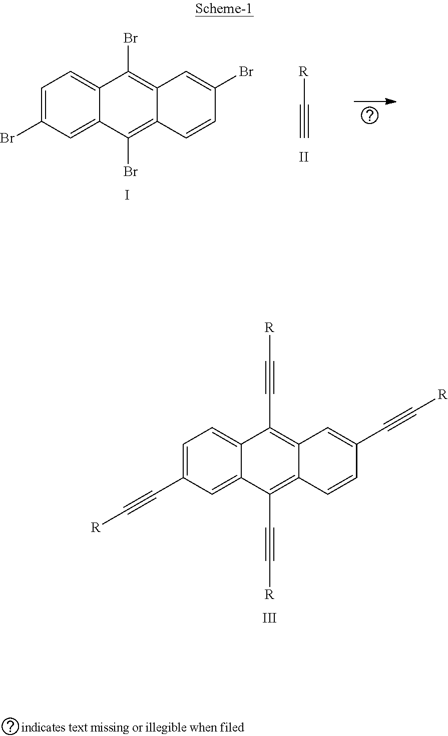

In another embodiment, the present invention provides compounds general Formula III which show positive solvatochrism and halochromism.

The above objects and advantages of the present invention will become apparent from the hereinafter set forth brief description of the drawings, detailed description of the invention, and claims appended herewith.

### BRIEF DESCRIPTION OF THE DRAWINGS

An understanding of the novel process of the present invention may be obtained by reference to the following drawings:

FIG. 1 depicts Interactions present in the solid-state structures of (a) 6 and (b) 6a according to an embodiment of the present invention.

FIG. 2 depicts Solvatochromism demonstrated by the compounds 6e and 6f according to an embodiment of the present invention.

FIG. 3 depicts Halochromism demonstrated by the compounds 6e and 6f;

FIG. 4 depicts the schematics of the fabricated sensing device using compound of Formula III as the channel material.

FIG. 5 depicts the I-V characteristic of the sensing device using compound of Formula III as the channel material.

FIG. 6 depicts the sensors' AER and sensitivity vs H₂ concentration plots.

FIG. 7 depicts the sensor's sensitivity to various VOCs at 900 ppm concentration.

FIG. 8 depicts the temperature response of the sensor.

FIG. 9 depicts the sensor's transient response.

### DETAILED DESCRIPTION OF THE INVENTION

The present invention now will be described hereinafter with reference to the detailed description, in which some, but not all embodiments of the invention are indicated. Indeed, the invention may be embodied in many different forms and should not be construed as limited to the embodiments set forth herein; rather, these embodiments are provided so that this disclosure will satisfy applicable legal requirements. Like numbers refer to like elements throughout. The present invention is described fully herein with non-limiting embodiments and exemplary experimentation.

The present invention provides a single step one pot route for synthesis of compound of Formula III via tetrafold sonogashira coupling.

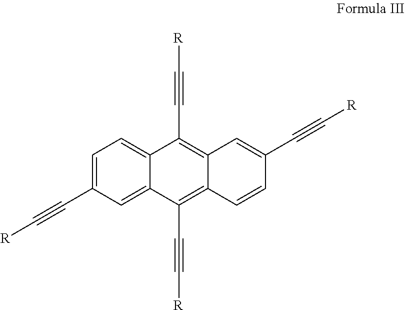

where R is hydrogen, alkyl, halo, aryl, substituted aryl, hetroaryl;
alkyl is selected from methyl, ethyl, isopropyl, cyclopentyl, cyclohexyl, amino, N—N dimethyl, trimethyl silyl, trifluoromethyl;
Aryl is selected from phenyl, Chloro phenyl, bromo phenyl, methyl benzene, ethyl benzene, methoxy benzene, N—N dimethyl aniline, toluly, 1-Napthyl, anthracenyl, biphenyl, benzophenone, triphenyl amine.
Heteroaryl group is selected from thiophene, furan, pyrole, pyridine, imidiazole, benzimidazole, quinolone, isoquinoline.
Halo is selected from chloro, Bromo, fluoro.

In an embodiment of the present invention is provided a tetra fold sonogashira coupling process for synthesis of compound of Formula III

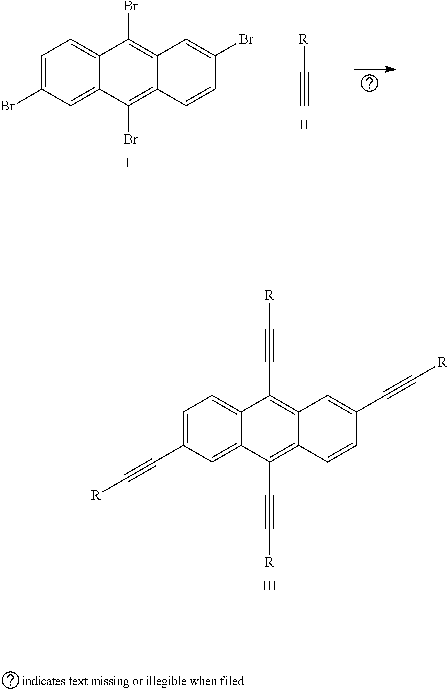

generally comprising the step of:
I. Degassing a 1:1 mixture of base and solvent under argon environment by freeze-pump-thaw process;
I. adding compound of Formula I, compound of Formula II, ligand, CuI and catalyst to the degassed reaction mixture obtained in step I; degassing the mixture for another 10-12 minutes;
III. refluxing the reaction mixture obtained in step II for 12-15 hours;
IV. passing the reaction through celite after completion of the reaction; and
V. evaporating the crude extract under reduced pressure to dryness and purifying the crude product by chromatography.
In an embodiment of the invention is described a process for synthesis of compounds with Formula III,

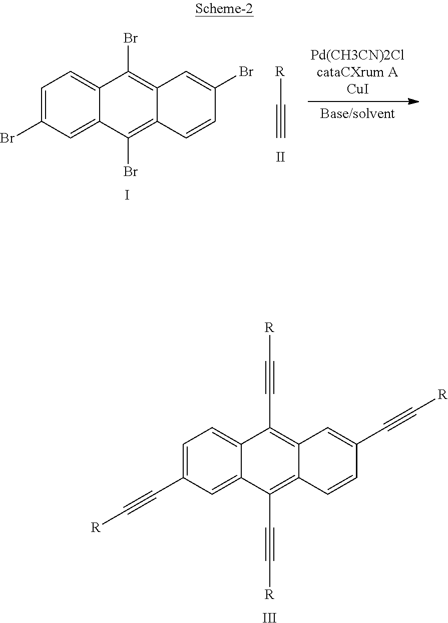

In an embodiment of the present invention, Scheme-2 utilizes a combination of palladium catalyst, Bis(acetonitrile)dichloropalladium(II) (Pd(CH₃CN)₂Cl₂) and ligand cataCXium®A in a ratio of 1:2.

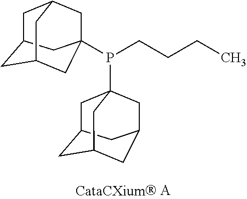 [SMILES: CCCCP(C12CC3CC(CC(C3)C1)C2)C12CC3CC(CC(C3)C1)C2]

In a preferred embodiment of the invention, the solvent used in the Scheme-2 is selected from tetrahydrofuran (THF), toluene, N-alkylpyrrolidones, dimethylformamide (DMF), Dioxane or mixture thereof.

In a preferred embodiment of the invention base is selected from trimethylamine, potassium carbonate, potassium bicarbonate, sodium bicarbonate, cesium carbonate, potassium hydroxide, piperidine. Sodium tert-butoxide, potassium tert-butoxide.

In a preferred embodiment of the invention the base and solvent are present in a ratio of 1:1 and the compound of Formula I and compound of Formula II are present in a ratio of 1:6.

In an embodiment of the invention are provided compounds with general Formula III prepared by the scheme III;

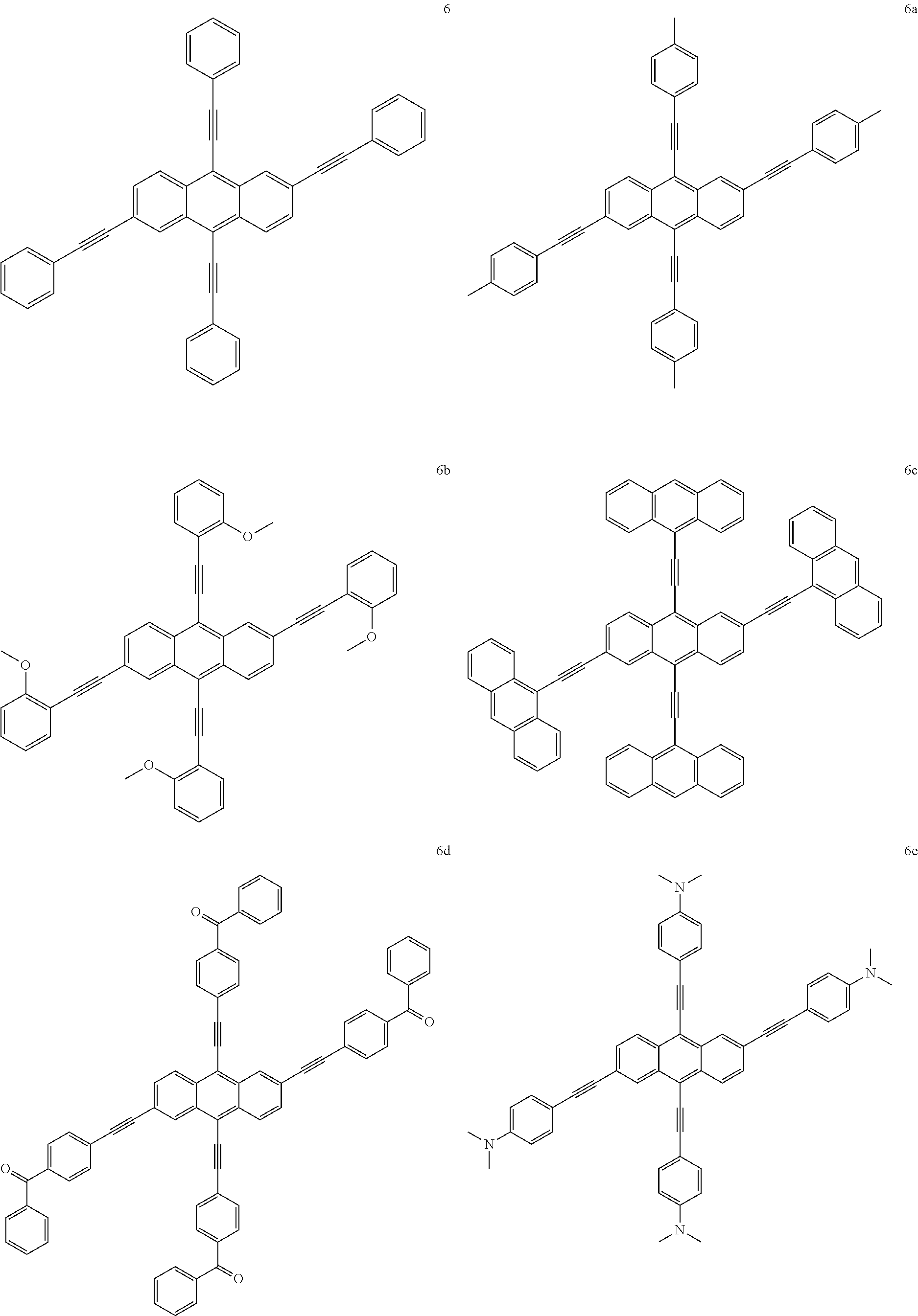

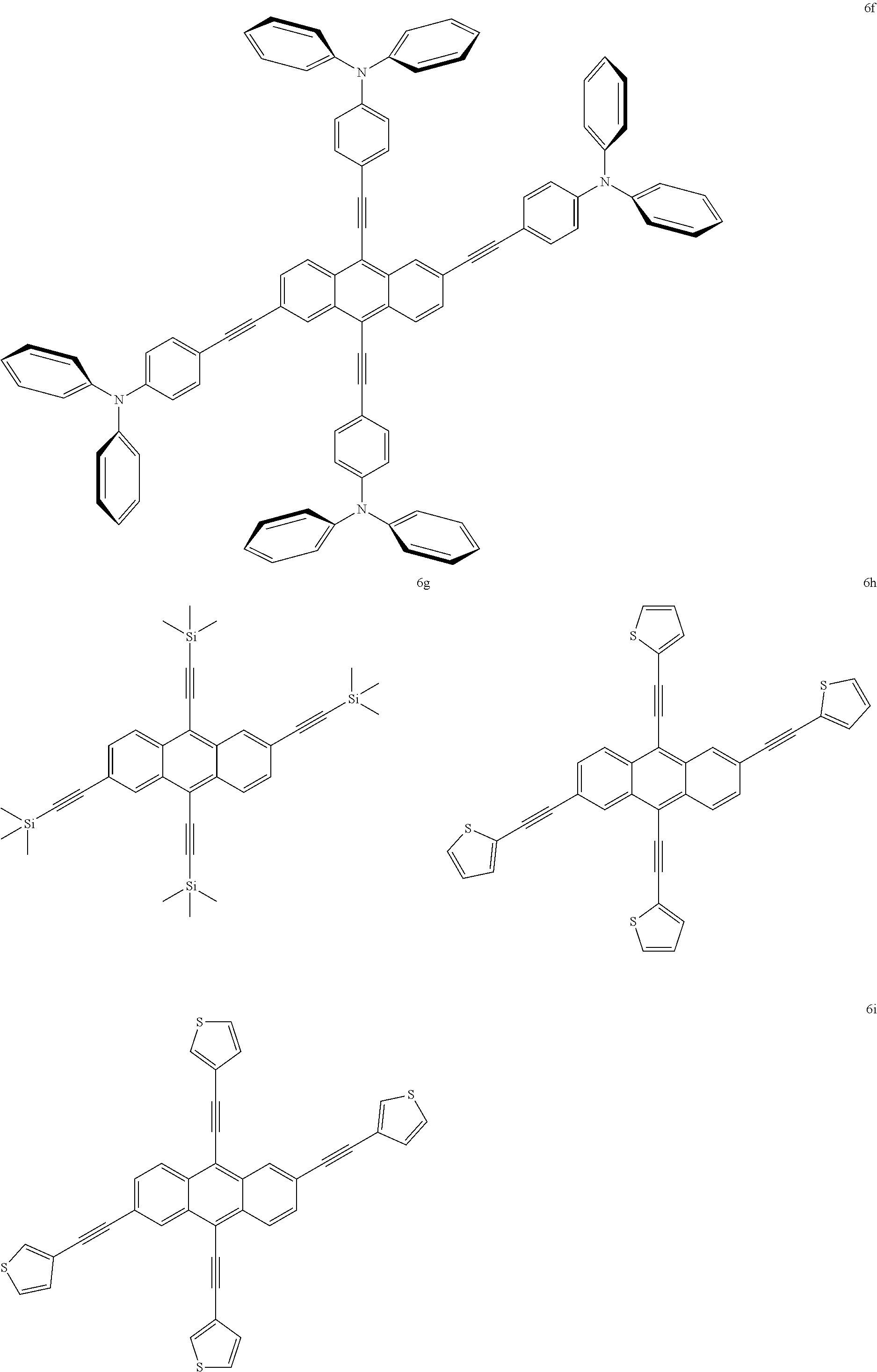

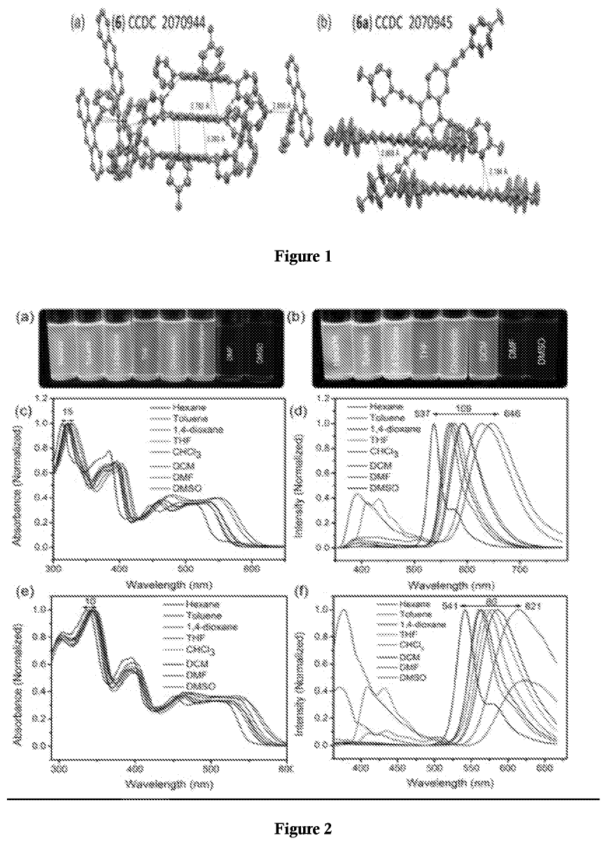

Referring to FIG. 1 of the present invention, there is illustrated interactions present in the solid-state structures of compound (a) 6 and (b) 6a. Compound 6 and 6a exhibit several p-p interactions apart from various C Phenyl-H . . . π interactions in the solid-state. The compound 6 shows two effective p-p interactions with an average distance of 3.393 Å (FIG. 1). Compound 6 demonstrates a total of seven C Phenyl-H . . . p(C≡C) interactions including two symmetrical bifurcated18 C-phenyl-H . . . π(C≡C) interactions with an average bond distance of 2.762 Å and five other unsymmetrical T-shaped C-phenyl-H . . . π(C≡C).

Referring to FIG. 2 of the present invention, there is illustrated the Solvatochromism demonstrated by the compounds 6e and 6f. Color in various solvents under 365 nm UV light is demonstrated by (a) 6e and (b) 6f. (c) Normalized absorption spectra of 6e according to Reichardt polarity scale by increasing the solvent polarity from hexane to DMSO. (d) Normalized emission spectra of 6e according to Reichardt polarity scale by increasing solvent polarity from hexane to DMSO. (c) Normalized absorption spectra of 6f according to Reichardt polarity scale by increasing the solvent polarity from hexane to DMSO. (d) Normalized emission spectra of 6f according to Reichardt polarity scale by increasing solvent polarity from hexane to DMSO.

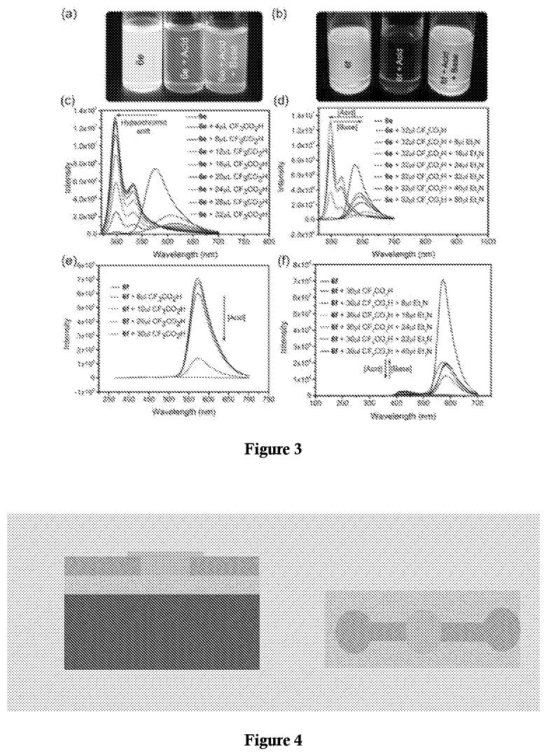

Referring to FIG. 3 of the present invention, there is illustrated Halochromism demonstrated by the compounds 6e and 6f, Color after sequential addition of acid and base under 365 nm UV light is demonstrated by (a) 6e and (b) 6f. (c) Hypsochromic shift in emission spectra of 6e upon gradual addition of CF3CO2H. (d) Major recovery of the emission spectra of 6e upon neutralization with Et3N. (c) Disappearance of emission maxima of 6f upon gradual addition of CF3CO2H. (d) Complete recovery of the emission spectra of 6f upon neutralization with Et3N.

Referring to FIG. 4 of the present invention, there is illustrated the schematic of the fabricated sensor having compound of Formula III as the channel material. The sensor was fabricated on a clean undoped silicon/silicon oxide (Si/SiO2) substrate that was exposed to UV light for 10 minutes before fabrication. A printing system (Make, Model: K-FAB TECH PRIVATE LIMITED, K-FT1PS) was used to control and optimise the metal contact pads' fabrication. The printing head of the system contains a stainless steel micro girder patterning tool through which ink flows down to the substrate. The printing was done at room temperature with a relative humidity of around 45 percent. On the SiO2 substrate, two distinct AgNP ink spots were drop-cast with a 1 mm spacing. The tip of the micro girder is positioned to lightly tap AgNP ink on the upper surface of a spot. AgNP ink is then dragged closer to the other spot to reduce the gap. The second AgNP spot is then treated similarly, with the ink dragged closer to the previously printed electrode to reduce the channel gap to about 20 μm. The printed electrodes were gently heated at 160° C. for 20 minutes. With 2 μl of AgNP ink, the entire electrode fabrication process is completed in 30 minutes. Further, the channel material i.e the compound of Formula III was dropped cast over the printed AgNP electrodes; the drop cast has a diameter of about 2 mm. The sensor was then annealed for five minutes at 120° C.

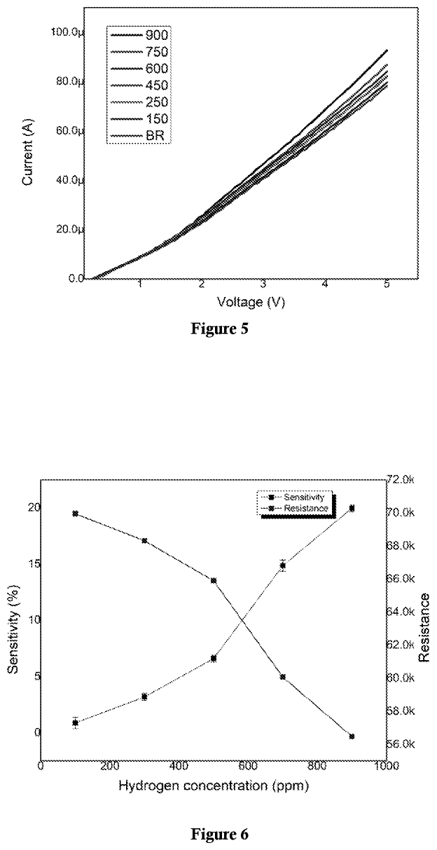

Referring to FIG. 5 of the present invention, there is illustrated the I-V characteristic of the sensing device using compound of Formula III as the channel material. The electrical characteristics of the sensing device were investigated using a Keithly 2600 source metre. A low-noise triaxial cable connects the device to the analyzer. For performing the sensing experiment, an in-house designed gas sensing system (Fabricator: Genrenew India) was integrated with the above analyzer via low-noise triaxial cables. The gas sensing chamber was outfitted with an Mass Flow Controller (MFC) panel that consists of three Mass Flow Controllers (MFCs) with varying flow rates to obtain the desired gas concentration. One MFC controls the carrier gas flow, while the other MFCs control the analyte gas flow. The sensing experiment was carried out in two circumstances: (a) in nitrogen ambient and (b) in different concentrations of hydrogen (H₂).

The gas sensing chamber was purged with nitrogen before being vacuumed three times to avoid cross-contamination. The sensors' baseline readings were taken in a nitrogen ambient, at less than 1% humidity and close to atmospheric pressure. Sweeping voltage from 0 to 5 V was used to analyse the I-V characteristic of sensing devices. The device shows a ohmic characteristic. A series of experiments were carried out to evaluate the response of the devices by varying the concentration of hydrogen gas from 100 to 9000 ppm. The electrical properties confirm an increase in current with increasing hydrogen concentration. The interaction with H₂ gas reduces the device's resistance. Because H₂ is a reducing gas, interacting with it raises the concentration of surface electrons on n type semiconductors. The following equation was used to determine the response of the gas sensors.

$$S = \frac{\vert {R}_{g} - {R}_{o} \vert}{{R}_{o}} \times 100 \%$$

where Rg is the average electrical resistance obtained by sweeping the voltage from 0 to 5V toward varying H₂ concentrations, and Ro is the average electrical resistance of the sensors in the nitrogen atmosphere.

Referring to FIG. 6 of the present invention, there is illustrated the sensors' AER and sensitivity vs H₂ concentration plots. The AER response confirms that a significant shift in the fabricated device. According to the results, the sensor has a high sensitivity of 19.95 percent to 900 ppm and 0.86 percent to 50 ppm.

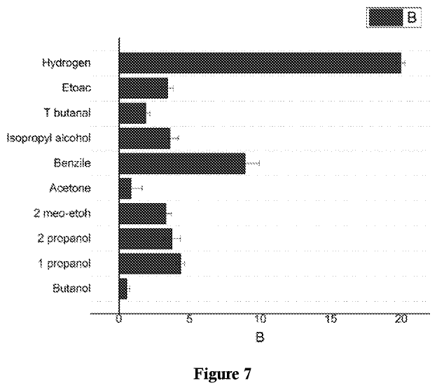

Referring to FIG. 7 of the present invention, there is illustrated the sensor's sensitivity to various VOCs at 900 ppm concentration. The desired VOC concentrations were achieved by combining it with DI water and injecting it into the chamber. VOCs exhibit significantly lower responses than H₂ gas at the same concentration. When the sensor's limit of detection (LOD) is calculated using the formula below [4], it shows a value of 49 ppm.

$$L O D = 3 \times \frac{\vert σ \vert}{slope}$$

where slope denotes the average electrical resistance for the various analyte gas concentrations and σ is the standard deviation of the base reading.

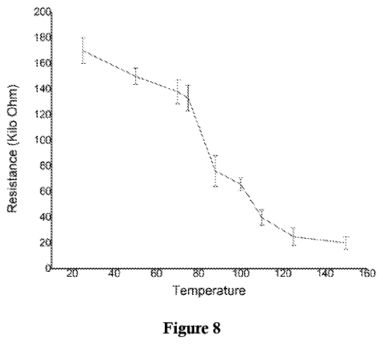

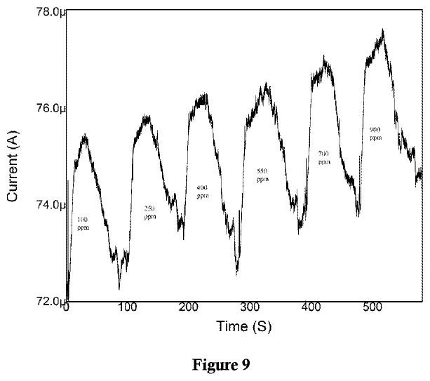

Referring to FIG. 8 of the present invention, there is illustrated the temperature response of the sensor. For the sensor's response to temperature variation, a tightly sealed chamber was used. Initially, the chamber was kept at 20° C. and then heated to 160° ° C. to analyze the sensor characteristics related to resistance variation. FIG. 8 depicts the variation of resistance with temperature. The resistance decreases significantly in the temperature range of 20 to 160 degrees Celsius. As a result, it is possible to conclude that the device's resistance decreases as the temperature rises. Temperature is an external factor that can affect the sensor.

Referring to FIG. 9 of the present invention, there is illustrated the sensor's transient response. The sensor was continuously flashed to seven pulses of H₂ gas at various concentrations of 100, 250, 400, 550, 700, and 900 ppm, accompanied by a mild evacuation of the gas in the sensing chamber at a constant voltage of 5 V. For different H₂ concentrations, the transient response provides an excellent response time ranging from 0 to 15 s with a good recovery time of 40 to 60 s. The gas in-conditioning process begins when the gas sensing chamber is pressurised to atmospheric pressure, and the gas out-conditioning process begins when the flow of the analyte gas is stopped and evacuated. A mild baseline shift was observed when the sensor shifted back and forth between mild vacuum and test gas environment.

TABLE 2
Photo physical properties of Tetraethynylated anthracenes synthesized via tetra-fold Sonogashira coupling

| | | | | λₑₘₛ (nm) | | | | | | | |
|---|---|---|---|---|---|---|---|---|---|---|---|
| | | λₘₐₓ^{a} | Log(ε)^{b} | | Thin | | | | E_{g}(eV)^{g} | | E_{HOMO}^{h} |
| Entry | Compound | (nm) | (LM⁻¹cm⁻¹) | Solution^{a} | Film^{c} | Powder^{d} | Φ(%)^{e} | τₐᵥ(ns)^{f} | Solution | Solid | (eV) |
| 1 | 6 | 345 | 4.17 | 500 | 543 | 601 | 61 | 4.54 | 2.47 | 2.04 | −5.78 |
| 2 | 6a | 348 | 3.92 | 507 | 595 | 584 | 61 | 4.36 | 2.44 | 2.00 | −5.58 |
| 3 | 6b | 359 | 4.03 | 514 | 633 | 653 | 60 | 4.83 | 2.39 | 1.95 | −5.33 |
| 4 | 6d | 368 | 3.74 | 517 | 610 | 636 | 47 | 2.96 | 2.36 | 1.92 | −2.84ⁱ |
| 5 | 6e | 322 | 3.54 | 575 | 642 | 402 | 31 | 4.18 | 2.16 | 1.79 | −5.11 |
| 6 | 6f | 344 | 4.07 | 572 | 635 | 639 | 31 | 3.56 | 2.21 | 1.99 | −5.27 |
| 7 | 6g | 310 | 4.05 | 464 | 556 | 567 | 60 | 6.70 | 2.62 | 1.92 | −5.39 |
| 8 | 6i | 342 | 3.95 | 503 | 547 | 567 | 56 | 4.79 | 2.47 | 2.04 | −5.71 |

$${τ}_{a v} = \frac{Σ {B}_{t} {τ}_{i}^{2}}{Σ {B}_{t} {τ}_{i}} ,$$

Referring to Table 2 of the present invention is illustrated, the photophysical properties of tetraethynylated anthracenes synthesized via tetra-fold Sonogashira coupling. ^{a}The absorption (10⁻⁵ M) as well as emission (10⁻⁶ M) spectra recorded in CHCl₃. ^{b} Log (ε) was calculated from the plot of absorbance vs concentration. ^{c}Emission spectra were recorded on glass on which a thin film was prepared by drop-cast method. ^{d}Emission spectra were recorded in powder form of the compounds. ^{e}Quantum yield were calculated with respect to quinine sulfate in 0.1 M H₂SO₄ as standard (φ=54%). ^{f}The average lifetime of the compounds calculated by using the equationwhere τₐᵥ=average lifetime in excited state of the luminescence compound, B=pre-exponential factor, τᵢ=decay lifetime of photoluminescence in excited state for the i^{th} component. ^{g}The band-gaps were calculated from the Tauc plot. determined from cyclic voltammetry. ⁱE_{LUMO} from cyclic voltammetry.

The quantum yield is the ratio between the emitted number of photons and the absorbed number of photons by the fluorophores. Relative fluorescence quantum yield was calculated by the following equation (1),

$$Φ\_F = Φ\_R \times I / I\_R \times A\_R / A \times ( η / η\_R ) 2 \qquad ( 1 )$$

Where ΦF and ΦR are relative quantum yields of analyte and reference respectively; I, IR are the area of emission of the analyte and reference; A, AR are the maximum absorbance of analyte and reference; η, ηR are the refractive index of analyte and reference solutions, respectively.

General Procedure for Synthesis of Compounds with Formula III:

A two-neck round bottom flask was taken, evacuated and charged with argon three times. anhydrous triethylamine (7 mL) and anhydrous THF (7 mL) were added into the round bottom flask and the solvent was degassed by freeze-pump-thaw process. This was followed by addition of 2, 6, 9, 10-tetrabromoanthracene (0.404 mmol), Pd(CH3CN)2Cl2 (0.0404 mmol), CataCXium® A (0.0810 mmol), CuI (0.0810 mmol), and alkyl/aryl acetylene (2.43 mmol) which were then stirred under reflux condition overnight. After the completion of the reaction, the reaction mixture was passed through celite by using DCM (200 mL×3) as eluent and filtrate was evaporated under reduced pressure. NMR yield of crude product was determined by ¹H NMR 1, 4-dioxane as external standard. The crude product was purified by column chromatography (eluent: Hexane and DCM).

### Example 1

### Synthesis of 2, 6, 9, 10-tetrakis(phenylethynyl)anthracene (6)

The compound was prepared following the general procedure for synthesis of compound of Formula III. The crude product was purified by column chromatography (eluent: 35% DCM in hexane). The product obtained was an orange solid with yield 58%. 1H NMR (400 MHZ, CDCl3) δ 8.79 (d, J=0.8 Hz, 2H), 8.60 (d, J=8.9 Hz, 2H), 7.69 (dd, J=6.2, 4.9 Hz, 5H), 7.66 (d, J=1.5 Hz, 1H), 7.53 (d, J=8.0 Hz, 4H), 7.28 (d, J=7.9 Hz, 4H), 7.20 (d, J=7.9 Hz, 4H). 2.45 (s, 6H), 2.40 (s, 6H). 13C{1H} NMR (151 MHz, CDCl3) δ 139.32, 138.88, 132.01, 131.87, 131.85, 131.67, 130.76, 129.51, 129.34, 127.63, 122.11, 120.26, 120.19, 118.45, 103.35, 91.88, 89.67, 85.52, 21.83, 21.75. MALDI-TOF calculated exact mass for C50H34 (M+): 634.26, found: 634.91.

### Example 2

### Synthesis of 2, 6, 9, 10-tetrakis((2-methoxyphenyl)ethynyl)anthracene (6b)

The compound was prepared following the general procedure for synthesis of compound of Formula III. The crude product was purified by column chromatography (eluent: 40% DCM in hexane) The product obtained was a red solid with yield 61%. 1H NMR (400 MHZ, CDCl3) δ 9.10 (s, 2H), 8.74 (d, J=8.9 Hz, 2H), 7.82-7.69 (m, 4H), 7.60 (dd, J=7.5, 1.6 Hz, 2H), 7.38 (ddd, J=15.6, 8.7, 1.6 Hz, 4H), 7.01 (ddd, J=26.0, 17.0, 8.0 Hz, 8H), 4.12 (s, 6H), 3.97 (s, 6H). 13C{1H} NMR (151 MHz, CDCl3) δ 160.73, 160.20, 133.81, 133.01, 132.19, 131.50, 131.39, 130.37, 130.11, 129.58, 127.67, 122.16, 120.71, 118.79, 112.85, 112.61, 110.87, 110.75, 99.87, 94.67, 91.01, 87.65, 56.00, 55.98. MALDI-TOF calculated exact mass for C50H34O4 (M+): 698.24, found: 698.65.

### Example 3

### Synthesis of ((anthracene-2, 6, 9, 10-tetrayltetrakis(ethyne-2, 1-diyl))tetrakis(benzene-4, 1-diyl))tetrakis(phenylmethanone) (6d)

The compound was prepared following the general procedure for synthesis of compound of Formula III. The crude product was purified by column chromatography (eluent: 90% DCM in hexane). The product obtained was a maroon solid with yield 88%. 1H NMR (400 MHZ, CDCl3) δ 8.87 (s, 2H), 8.67 (d, J=8.9 Hz, 2H), 7.94 (s, 8H), 7.84 (dd, J=14.4, 7.7 Hz, 12H), 7.77 (t, J=9.2 Hz, 6H), 7.63 (q, J=7.2 Hz, 4H), 7.53 (q, J=7.4 Hz, 8H). 13C{1H} NMR (151 MHz, CDCl3) δ 196.06, 195.99, 137.71, 137.50, 137.45, 137.34, 132.90, 132.80, 132.21, 132.04, 131.83, 131.81, 131.19, 130.50, 130.33, 130.19, 130.15, 129.88, 128.62, 128.57, 127.78, 127.30, 127.15, 122.10, 118.67, 102.82, 92.87, 91.44, 88.66. MALDI-TOF calculated exact mass for C74H₄₂₀₄ (M+): 994.30, found: 994.86.

### Example 4

### Synthesis 4, 4′, 4″,4′″-(anthracene-2, 6, 9,10-tetrayltetrakis(ethyne-2, 1-diyl))tetrakis(N, N-dimethylaniline) (6e)

The compound was prepared following the general procedure for synthesis of compound of Formula III. The crude product was purified by column chromatography (eluent: 60% DCM in hexane). The product obtained was a brown solid with yield 64%. 1H NMR (400 MHZ, CDCl3) δ 8.78 (s, 2H), 8.60 (d, J=8.8 Hz, 2H), 7.68 (d, J=8.7 Hz, 4H), 7.64 (d, J=8.1 Hz, 2H), 7.52 (d, J=8.7 Hz, 4H), 6.78 (d, J=8.9 Hz, 4H), 6.70 (d, J=8.8 Hz, 4H), 3.06 (s, 12H), 3.02 (s, 12H). 13C{1H} NMR (151 MHZ, CDCl3) δ 150.55, 150.34, 133.13, 133.11, 131.85, 131.34, 130.25, 129.25, 127.59, 122.19, 118.19, 112.12, 112.01, 110.37, 110.25, 104.33, 92.70, 88.78, 84.84, 40.42, 40.39. MALDI-TOF calculated exact mass for C54H46N4 (M+): 750.37, found: 750.90.

### Example 5

### Synthesis of 4, 4′, 4″, 4″-(anthracene-2, 6, 9, 10-tetrayltetrakis(ethyne-2, 1-diyl))tetrakis(N, N-diphenylaniline) (6f)

The compound was prepared following the general procedure for synthesis of compound of Formula III. The product obtained was a maroon solid with yield 31%. 1H NMR (400 MHZ, CDCl3) δ 8.76 (d, J=0.8 Hz, 2H), 8.58 (d, J=8.9 Hz, 2H), 7.67-7.61 (m, 6H), 7.46 (d, J=8.7 Hz, 4H), 7.29 (dt, J=9.7, 4.9 Hz, 15H), 7.15 (dd, J=14.7, 8.0 Hz, 18H), 7.07 (ddd, J=21.2, 9.4, 5.3 Hz, 15H). 13C{1H} NMR (151 MHZ, CDCl3) δ 148.61, 148.27, 147.30, 147.23, 132.91, 132.88, 131.96, 131.56, 130.58, 129.62, 129.56, 129.42, 127.61, 125.31, 125.21, 123.91, 123.77, 122.35, 122.11, 118.28, 116.00, 115.97, 103.60, 92.03, 89.70, 85.64. MALDI-TOF calculated exact mass for C94H62N4 (M+): 1247.50, found: 1247.97.

### Example 6

### Synthesis of (anthracene-2, 6, 9, 10-tetrayltetrakis(ethyne-2, 1-diyl))tetrakis(trimethylsilane) (6g)

The compound was prepared following the general procedure for synthesis of compound of Formula III. The crude product was purified by column chromatography (eluent: hexane). The product obtained was a yellow solid with yield 83%. 1H NMR (600 MHz, CDCl3) δ 8.66 (s, 2H), 8.44 (d, J=8.9 Hz, 2H), 7.57 (dd, J=8.9, 1.3 Hz, 2H), 0.43 (s, 18H), 0.31 (s, 18H). 13C{1H} NMR (151 MHz, CDCl3) δ 132.16, 131.82, 131.77, 129.55, 127.48, 121.92, 118.47, 109.46, 105.59, 100.92, 96.83, 0.22, 0.10. MALDI-TOF calculated exact mass for C34H₄₂Si4 (M+): 562.23, found: 562.62.

### Example 7

### Synthesis of 2, 2′, 2″, 2″-(anthracene-2, 6, 9, 10-tetrayltetrakis(ethyne-2, 1-diyl)) tetrathiophene (6h)

The compound was prepared following the general procedure for synthesis of compound of Formula III. The crude product was purified by column chromatography (eluent: 40% DCM in hexane). The product obtained was a yellow powder with yield 59%. 1H NMR (600 MHZ, CDCl3) δ 8.73 (s, 2H), 8.55 (d, J=8.9 Hz, 2H), 7.68 (d, J=8.9 Hz, 2H), 7.56 (d, J=3.5 Hz, 2H), 7.45 (d, J=5.1 Hz, 2H), 7.40 (d, J=3.4 Hz, 2H), 7.36 (d, J=5.1 Hz, 2H), 7.17-7.13 (m, 2H), 7.08-7.05 (m, 2H). 13C{1H} NMR (151 MHz, CDCl3) δ 132.94, 132.65, 131.88, 131.65, 130.61, 129.43, 128.53, 127.98, 127.67, 127.64 127.40, 123.27, 123.09, 121.93 118.33, 96.50, 93.82, 89.80, 85.26. MALDI-TOF calculated exact mass for C38H₁₈S4 (M+): 602.02, found: 602.36.

### Example 8

### Synthesis of 3, 3′, 3″, 3″-(anthracene-2, 6, 9, 10-tetrayltetrakis(ethyne-3, 1-diyl)) tetrathiophene (6i)

The compound was prepared following the general procedure for synthesis of compound of Formula III. The crude product was purified by column chromatography (eluent: 40% DCM in hexane). The product obtained was a brown powder with yield 20%. 1H NMR (400 MHZ, CDCl3) δ 8.78 (s, 2H), 8.60 (d, J=8.9 Hz, 2H), 7.81 (dd, J=2.7, 1.3 Hz, 2H), 7.69 (d, J=1.6 Hz, 1H), 7.66 (d, J=1.5 Hz, 1H), 7.65-7.63 (m, 1H), 7.46-7.44 (m, 4H), 7.35 (dd, J=5.0, 3.0 Hz, 2H), 7.30 (dd, J=5.1, 0.9 Hz, 3H). 13C{1H} NMR (151 MHz, CDCl3) δ 132.00, 131.70, 130.74, 130.16, 130.08, 129.65, 129.50, 129.39, 127.65, 126.00, 125.71, 122.25, 122.21, 121.96, 118.37, 98.16, 89.64, 86.90, 85.47. MALDI-TOF calculated exact mass for C46H₂₆ (M+): MALDI-TOF calculated exact mass for C38H18S4 (M+): 602.02, found: 602.41

TABLE 1
Physical appearance and yield of synthesized
symmetric tetraethynylated anthracenes

| Compound | Yield in %(¹H-NMR | Physical |
|---|---|---|
| Code | conversion) | appearance |
| 6 | 98 | Orange solid |
| 6a | 92 | Orange solid |
| 6b | 84 | Orange solid |
| 6c | NA | — |
| 6d | 93 | Red solid |
| 6e | 76 | Brown solid |
| 6f | 65 | Maroon solid |
| 6g | 64 | Yellow solid |
| 6h | 69 | Yellow solid |
| 6i | 88 | Brown solid |

Referring to Table 1 of the present invention, there is illustrated the yields and physical appearance of the compounds synthesized via tetra-fold Sonogashira coupling.

Therefore, the present invention provides a robust, efficient and a single step one pot process for synthesis of symmetric tetraethynylated compounds which show exciting photophysical properties where these symmetric tetraethynylated anthracenes exhibit solvatochromism and halochromism. The former also exhibits a low band-gap of 1.79 eV in the solid-state. The present invention synthesizes the symmetric tetraethynylated compounds in good to excellent yield.

Many modifications and other embodiments of the invention set forth herein will readily occur to one skilled in the art to which the invention pertain having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

We claim:

1. A symmetric tetraalkynylated anthracene derivative having general Formula III: 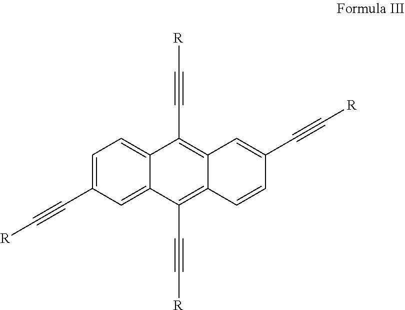
wherein:
R is selected from but not limited to hydrogen, alkyl, halo, aryl, substituted aryl, hetroaryl;
an alkyl is selected from but not limited to methyl, ethyl, isopropyl, cyclopentyl, cyclohexyl, amino, N—N dimethyl, trimethyl silyl, trifluoromethyl;
an aryl is selected from but not limited to phenyl, chloro phenyl, bromo phenyl, methyl benzene, ethyl benzene, methoxy benzene, N—N dimethyl aniline, toluly, 1-Napthyl, anthracenyl, biphenyl, benzophenone, triphenyl amine;
a heteroaryl group is selected from but not limited to thiophene, furan, pyrole, pyridine, imidiazole, benzimidazole, quinolone, isoquinoline;
a halo is selected from but not limited to chloro, Bromo, fluoro;
said anthracene derivative of Formula III is a channel material for sensors and optoelectronic devices; and
wherein said symmetric tetraethynylated anthracenes show positive solvatochrism and halochromism.

2. The symmetric tetraalkynylated anthracene derivatives as claimed in claim 1, wherein the derivatives with Formula III are selected from but not limited to

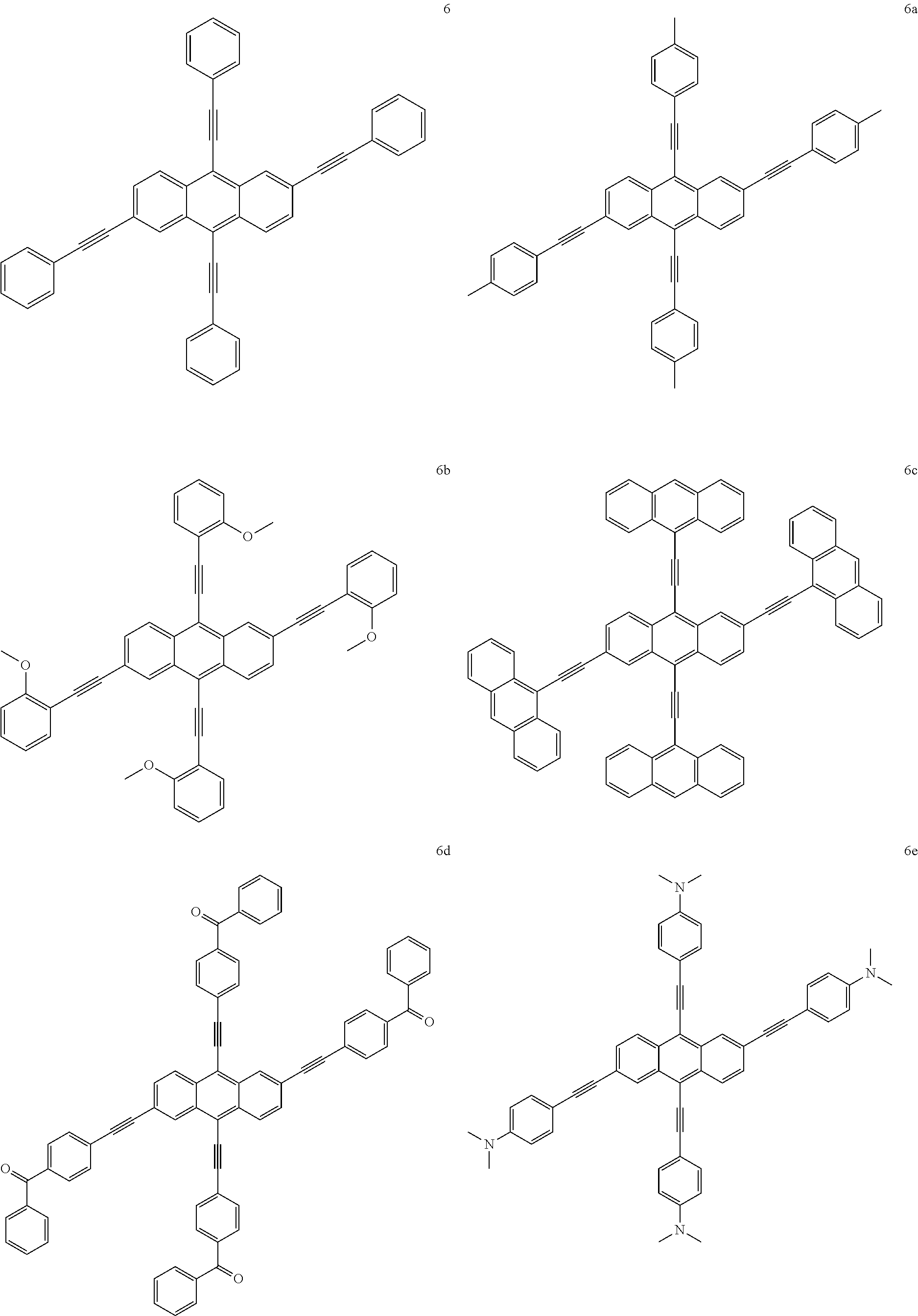

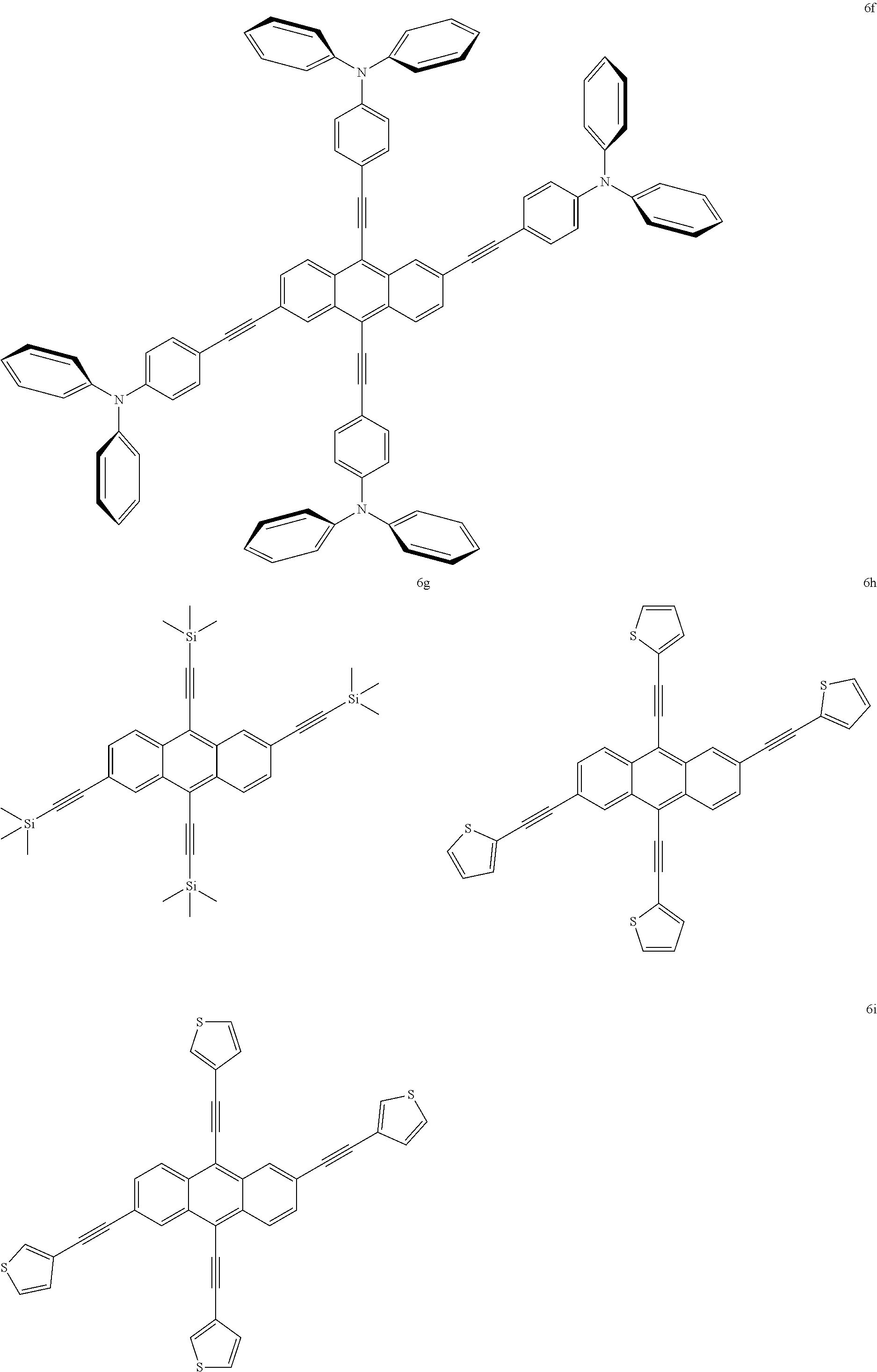

3. The sensor comprising symmetric tetraalkynylated anthracene derivatives as claimed in claim 1 as channel material.

4. The sensor as claimed in claim 3, wherein the sensor has a high sensitivity of 19.95 percent to 900 ppm and 0.86 percent to 50 ppm.

5. The sensor as claimed in claim 3, wherein the sensor has an excellent response time ranging from 0 to 15 s with a good recovery time of 40 to 60 s.

6. A process for synthesizing symmetric tetraalkynylated anthracene derivatives having Formula III according to Scheme-2: 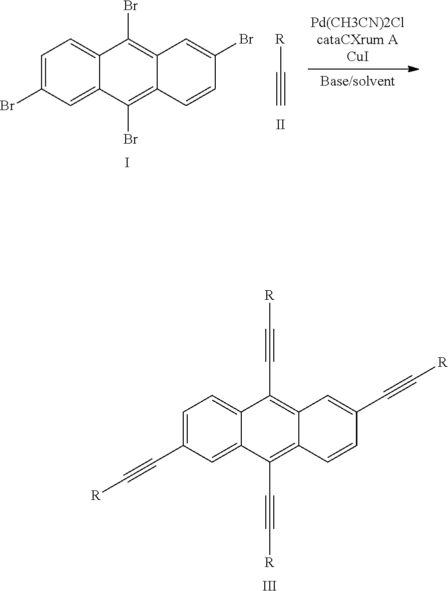
wherein the process comprising the steps of:
a) degassing a 1:1 mixture of base and solvent under argon environment by freeze-pump-thaw process;
b) adding compound of Formula I, compound of Formula II, ligand, CuI and catalyst to the degassed reaction mixture obtained in step I and further degassing the mixture for another 10-12 minutes;
c) refluxing the degassed reaction mixture obtained in step II for 12-15 hours;
d) passing the reaction mixture obtained in step III through celite after completion of the reaction to obtain a crude extract; and
e) evaporating the crude extract under reduced pressure to dryness and purifying the crude product by chromatography to obtain compound of Formula III.

7. The process as claimed in claim 6, wherein a combination of palladium catalyst, Bis(acetonitrile)dichloropalladium(II) (Pd(CH3CN)2Cl2) and ligand (cataCXium A) is used in a ratio of 1:2.

8. The process as claimed in claim 6, wherein the base and solvent are present in a ratio of 1:1 and the compound of Formula I and compound of Formula II are present in a ratio of 1:6.

9. The process as claimed in claim 6, wherein the base is selected from but not limited to trimethylamine, potassium carbonate, potassium bicarbonate, sodium bicarbonate, cesium carbonate, potassium hydroxide, piperidine, sodium tert-butoxide, potassium tert-butoxide.

10. The process as claimed in claim 6, wherein the solvent is selected from but not limited to tetrhydrofuran (THF), toluene, N-alkylpyrrolidones, dimethylformamide (DMF), dioxane or mixture thereof.
